(19) United States
(12) Patent Application Publication
(10) Pub. No.: US 2005/0214259 A1
(43) Pub. Date: Sep. 29, 2005

(54) Corneal endothelium-like sheet and method of constructing the same

(76) Inventors: Yoichiro Sano, Kyoto (JP); Shigeru Kinoshita, Osaka (JP); Yutaka Ishino, Kyoto (JP); Takahiro Nakamura, Kyoto (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 10/510,958

(22) PCT Filed: Feb. 13, 2003
(86) PCT No.: PCT/JP03/01515

§ 371 Date: Oct. 29, 2004

(30) Foreign Application Priority Data
Apr. 30, 2002 (JP) 2002-129238

(51) Int. Cl. C12N 5/08; A61K 45/00

(52) U.S. Cl. 424/93.7; 435/371; 435/368

(57) ABSTRACT

It is our intention to provide a transplantation material applicable to the treatment of various diseases that require corneal endothelium transplantation. Corneal endothelial cells are collected, proliferated and then sown on amnion and cultured. Thus, a corneal endothelium-like sheet, which has a cell layer made up of the corneal endothelium-origin cells formed on the amnion, can be obtained.

### TECHNICAL FIELD

The present invention relates to corneal endothelium-like sheets and methods of constructing the same. The corneal endothelium-like sheet provided by the present invention can be used as a transplantation material for treating diseases that require corneal endothelial transplantation such as bullous keratopathy, corneal edema, keratoleukoma and keratoconus.

### BACKGROUND ART

In recent years, the proliferation in the number of intraocular operations has rapidly increased the number of postoperative diseases in which irreversible disorders occur in the corneal endothelium, resulting in corneal opacity. At the present time, the treatment used for such diseases in the corneal endothelium is penetrating keratoplasty. However, postoperative rejection of the endothelium following penetrating keratoplasty, is one of the largest problems in the clinical field of ophthalmology, and the development of a new technique to help prevent rejection has eagerly been awaited.

Meanwhile, the normal anterior ocular segment is well known to be an immune privileged site concerned with anterior chamber associated immune deviation. Factors constituting this immunosuppressive environment; immunosuppressive agents such as transforming growth factor beta (TGF-β), vasoactvie intestinal peptide (VIP), which are produced in the cornea and the iris, play an important role. Furthermore, it is reported that the production of immunosuppressive agents from the anterior ocular segment is closely related to the corneal sensory nerve running through the corneal stroma and that production of the immunosuppressive agent is suppressed when the corneal sensory nerve is cut during penetrating keratoplasty (Streilein J W, Bradley D, Sano Y, Sonoda Y: Immunosuppressive properties of tissues obtained from eyes with experimentally manipulated corneas. Invest Ophthalmol Vis Sci. 37(2):413-424, 1996. Sano Y, Streilein J W: Effects of corneal surgical wounds on ocular immune privilege. In: Nussenblatt R B, Whitcup S M, Caspi R R, and Gery I eds. Advances in Ocular immunology. Amsterdam, ELSEVIER:207-210, 1994.).

Therefore, in those corneal endothelial diseases for which penetrating keratoplasty is performed in order to minimize the failure of the immunosuppressive environment of the anterior ocular segment, if only damaged endothelial cells can be replaced by healthy endothelial cells, it becomes an ideal operating technique as one can expect rejection to be suppressed. Also, since only the posterior surface of the cornea is replaced, corneal astigmatism occurs less frequently after operation, so recovery of visual acuity from an early stage is expected.

Since a substrate as a carrier is essential for the transplantation of corneal endothelial cells, it is necessary to produce a sheet in which corneal endothelial cells are layered on the substrate in vitro. Conventionally, it has been thought that a corneal endothelial cell does not have proliferation potency, but proliferation has been confirmed in vitro (Hyldahl L: Primary cell cultures from human embryonic corneas. J Cell Sci. 66: 343-351, 1984. Senoo T, Obara Y, Joyce N: EDTA: A promoter of proliferation in human corneal endothelium. Invest Ophthalmol Vis Sci. 41: 2930-2935, 2000. Miyata K, Drake J, Osakabe Y, et al.: Effect of donor age on morphologic variation of cultured human corneal endothelial cells. Cornea. 20: 59-63, 2001).

Therefore, it is thought that if an appropriate substrate and culture condition are found, a corneal endothelial cell sheet can be produced. On the other hand, it has been suggested that stem cells are present in the peripheral portion of the corneal endothelial cell layer (Bednarz J, Engelmann K: Indication for precursor cells in the adult human corneal endothelium. Invest Ophthalmol Vis Sci. 42(suppl): S274, 2001). So, if corneal endothelium stem cells with high proliferation potency can be cultured and proliferated in vitro, from a small number of corneal endothelial cells, it may be possible to produce a corneal endothelial sheet which can be used as a transplantation material. In particular, if a residual autologous corneal endothelial cell is used, a rejection-free corneal transplantation can probably be developed.

The present invention has been conceived under the above-mentioned circumstances. The aim of this invention is to provide a corneal endothelium-like sheet that can be used for corneal endothelial transplantation to treat corneal endothelial diseases.

### DISCLOSURE OF INVENTION

The present inventors have investigated earnestly in view of the above-mentioned conditions. Specifically, they have investigated the selection of a substrate suitable for culturing corneal endothelial cells in vitro and a method for forming cell layers with a high cell density. Firstly, corneal endothelial cells were collected, and then cultured and proliferated in vitro. A cell suspension with high cell density was produced by subculturing the proliferated cells and subjecting them to appropriate centrifugation. Then, as a substrate (carrier), amniotic membrane containing collagen as a main component was employed, and the cell suspension was planted thereon and cultured for a predetermined time. As a result, a single layered cell layer, in which cells derived from the corneal endothelial cells have a similar morphology to that of the living body, was formed. When this cell layer was observed, it had the equivalent cell density to the corneal endothelial cells of a living body and had a configuration in which hexagonal shaped cells were regularly layered to form a single layer structure. On the other hand, when the resultant composition sheet consisting of amniotic membrane and a cell layer was transplanted in a rabbit from which a part of the corneal endothelial cell layer had been removed, good survival was observed. It was confirmed that the composition could be suitably used as a material for corneal endothelial cell transplantation. The present invention was completed based on the above-mentioned findings and provides the following configurations.

[1] A corneal endothelium-like sheet comprising: a collagen layer and a cell layer formed on the collagen layer; the cell layer consisting of cells derived from corneal endothelium.

[2] A corneal endothelium-like sheet described in [1], wherein the collagen layer is derived from amniotic membrane.

[3] A corneal endothelium-like sheet described in [1], wherein the collagen layer consists of amniotic membrane from which the epithelium has been removed.

[4] A corneal endothelium-like sheet comprising a cell layer consisting of cells derived from corneal endothelium.

[5] A corneal endothelium-like sheet described in any of [1] to [4], wherein the cell layer has a monolayer structure.

[6] A corneal endothelium-like sheet described in any of [1] to [5], wherein the cell density of the cell layer is about 2000 cells/mm² to about 4000 cells/mm².

[7] A corneal endothelium-like sheet described in any of [1] to [6], wherein the plane view shape of the cells derived from corneal endothelium is substantially hexagonal.

[8] A corneal endothelium-like sheet described in any of [1] to [7], wherein the cells derived from corneal endothelium are arranged regularly in the cell layer.

[9] A method for constructing a corneal endothelium-like sheet, the method comprising the following steps:
a) culturing and proliferating collected corneal endothelial cells;
b) collecting the proliferated corneal endothelial cells and producing a cell suspension;
c) planting the cell suspension on a collagen layer and culturing thereof.

[10] A method for producing a corneal endothelium-like sheet described in [9], wherein the following step is carried out after step b):
b-1) increasing the cell density in the cell suspension using centrifugation.

[11] A method for producing a corneal endothelium-like sheet described in [9] or [10], wherein centrifugation is carried out after planting the cell suspension in step c).

[12] A method for producing a corneal endothelium-like sheet described in [9] or [10], wherein step c) comprises the following procedures:
c-1) placing a container in a culture container, the container having a bottom surface consisting of a membrane with a pore size capable of allowing a culture solution to pass through;
c-2) forming acollagen layer on the bottom face of the container;
c-3) planting the cell suspension on the collagen layer;
c-4) carrying out centrifugation;
c-5) culturing.

[13] A method for producing a corneal endothelium-like sheet described in any of [9] to [12], wherein the collagen layer is derived from amniotic membrane.

[14] A method for producing a corneal endothelium-like sheet described in any of [9] to [12], wherein the collagen layer consists of amniotic membrane from which the epithelium has been removed.

Note that the “corneal epithelium-like sheet” herein is used as a term for meaning a composition that has a similar feature to a corneal endothelial cell layer and can be transplanted in order to reconstruct a corneal endothelial cell layer. Furthermore, unless otherwise specified, “corneal endothelial cell” is used as a term encompassing cells included in a corneal endothelial cell layer. That is to say, it also includes corneal endothelial stem cells.

### BRIEF DESCRIPTION OF DRAWINGS

FIG. 1 shows the morphology of a corneal endothelium like cell layer formed on amniotic membrane from which the epithelium has been removed. A shows an HE (hematoxylin-eosin) stained image; B shows an alizarin stained image; and C and Dare scanning electron microscope images.

FIG. 2 shows slit photographs of an anterior ocular segment at day 4 after transplantation via full thickness trepanation. A shows a control group (a group in which the Descemet's membrane has been removed); B shows a group with amniotic membrane (AM group); and C shows a group with a corneal endothelium-like sheet.

FIG. 3 shows slit photographs of an anterior ocular segment at day 7 after transplantation via full thickness trepanation. A shows a control group (a group in which the Descemet membrane has been removed); B shows an AM group; and C shows a group using a corneal endothelium-like sheet.

FIG. 4 is a graph showing the changes in corneal thickness before and after transplantation via full thickness trepanation. In this graph, * represents p<0.05. In the group where a corneal endothelium-like sheet was used, the corneal thickness is reduced significantly (p<0.05) as compared with the control group and the AM group. FIG. 5 is a cross-sectional view schematically showing the state of the instruments when endothelial cells are cultured. In a culture dish (1), a culture insert (2) is disposed. Furthermore, it shows the state in which the corneal endothelial cells (3) are cultured on the bottom face of the culture insert (2). Reference numeral 5 denotes a culture medium.

FIG. 6 is a cross-sectional view schematically showing the state of the instruments when endothelial cells are cultured on amniotic membrane. In a culture dish (1), a culture insert (2) is disposed. It also shows the situation in which amniotic membrane (4) is extended on the bottom face of the culture insert (2) and cells (3) derived from the corneal endothelial cells are cultured thereon. Reference numeral 5 denotes a culture medium.

1: culture dish, 2: culture insert (container for inserting culture), 3: cells derived from the corneal endothelial cell, 4: amniotic membrane, 5: culture medium.

### BEST MODE FOR CARRYING OUT THE INVENTION

The present invention relates to a corneal endothelium-like sheet comprising a collagen layer, and a cell layer formed on the collagen layer, the cell layer consisting of cells derived from corneal endothelium.

Herein, the types of collagen constituting the collagen layer are not limited in particular, but it is preferable for the collagen layer to be derived from amniotic membrane. Furthermore, it is also preferable to use amniotic membrane from which the epithelium has been removed by such methods as scraping treatment. Whether or not the collagen layer is made of amniotic membrane from which the epithelium has been removed can be confirmed by examining to check that a cell from the amniotic membrane epithelium layer is not contained in the collagen layer. It is preferable that human amniotic membrane is used. Note that “derived from amniotic membrane” broadly means that amniotic membrane is used as a starting material.

On the other hand, “cells derived from corneal endothelium” means cells produced by culturing the collected corneal endothelial cells, so that they are proliferated and differentiated.

It is preferable for the corneal endothelial like sheet to have some, or more suitably, all of the characteristics or features mentioned below.

(1) The cell layer has a monolayer structure (single-layered structure). This is one of the features of the corneal endothelial cell layer in the living body.

(2) The cell density of the corneal endothelial cell layer of the living body is said to be about 4000 cells/mm² in a newborn human. It decreases in accordance with growth and aging and reaches about 2000 cells/mm² to about 3000 cells/mm² in adult humans in the normal state. In view of this fact, it is preferable for the cell density of the cell layer constituting the corneal endothelium-like sheet presented in this invention, to be about 2000 cells/mm² to about 4000 cells/mm². In particular, in a case where a recipient (a person to be transplanted) is an adult, it is preferable for the cell density to be about 2000 cells/mm² to about 3000 cells/mm².

(3) The plane view shape of cells constituting the cell layer is hexagonal. This is one of the features of the corneal endothelial cells in the living body. When this feature is observed, it is expected that the corneal endothelium-like sheet should be similar to the corneal endothelium cell layer and should exhibit similar functions to those of the corneal endothelial cell layer.

(4) In the cell layer, cells are regularly arranged. It is thought that in the corneal endothelial cell layer of the living body, cells constituting the corneal endothelial cell layer are regularly arranged, so that high transparency is maintained and the moisture control function of the cornea is exhibited appropriately. Therefore, when such a morphological feature is provided, the corneal endothelium-like sheet in this present invention should exhibit similar functions to those of the corneal endothelial cell layer in the living body.

The corneal endothelium-like sheet in this present invention can be produced, for example, by the following method.

<1> The Collection and Proliferation of Corneal Endothelial Cells.

Corneal endothelial cells are collected from the cornea of the recipient him/herself or from an appropriate donor, in the usual manner. For example, the Descemet's membrane and the endothelial cell layer of the corneal tissue are peeled off from the corneal stroma, then transferred to a culture dish and treated with dispase. Thus, corneal endothelial cells are peeled away from the Descemet's membrane. The corneal endothelial cells residing in the Descemet's membrane can be peeled of f by such means as pipetting. The Descemet's membrane is removed, after which, corneal endothelial cells are cultured in an appropriate culture solution. For a culture solution, it is possible to use, for example, a commercially available DMEM (Dullbecco's Modified Eagle's Medium) to which FBS (fetal bovine serum), b-FGF (basic-fibroblast growth factor), EGF (epidermal growth factor), insulin and antibiotics such as penicillin and streptomycin are added. It is preferable to use a culture container (culture dish) the surface of which is coated with type I collagen, type IV collagen, fibronectin, laminin or extracellular matrix of bovine endothelial cells. This is advantageous because the attachment of the corneal endothelial cells to the surface of the culture container is promoted, thus enabling excellent proliferation.

The temperature conditions for culturing corneal endothelial cells are not necessarily limited as long as the corneal endothelial cells can grow. However, the temperature should be for example, about 25° C. to about 45° C. When considering proliferation efficiency, a preferable temperature is about 30° C. to 40° C.; more specifically, 37° C. The culture time varies depending upon the state of cells to be used and so on but generally, it is 7 to 14 days.

It is preferable to use corneal endothelial cells from the recipient him/herself, if possible. This is advantageous because a corneal endothelium-like sheet, that may be free from immunorejection when it is employed for transplantation, can be produced. Thus transplantation that is not accompanied by immunorejection can be performed. When it is impossible or difficult to obtain corneal endothelial cells from the recipient him/herself, corneal endothelial cells from a person other than the recipient may be used. However, in this case, it is preferable to select a donor by considering immunocompatibility.

<2> Subculture and Production of Cell Suspension.

After the corneal endothelial cells used for culture are proliferated, a subculture of the cells can be carried out. Preferably, a subculture is carried out when cells become subconfluent or confluent. The subculture can be carried out as follows. Firstly, cells are peeled off from the surface of the culture container by treating with trypsin-EDTA. Then, cells are collected. Culture solution is added to the collected cells to create a cell suspension. It is preferable for centrifugation to be carried out when or after cells have been collected. With such centrifugation, cell suspension with a high cell density can be prepared. Note here that conditions for centrifugation can include 500 rpm (30 g) to 1000 rpm (70 g) and 1 to 10 minutes.

As in the above-mentioned initial culture, a cell suspension is planted on the culture container and cultured. The subculture can be carried out in the same culture conditions as the above-mentioned initial culture. The culture time is different depending upon the state of the cells to be used, but generally, it is 7 to 21 days. The above-mentioned subculture can be carried out a number of times if necessary. By repeating the subcultures, the number of cells can be increased and a cell suspension with a high cell density can be prepared. Finally, it is preferable to prepare a cell suspension with a cell density of about 5×10⁵ cells/ml to 20×10⁵ cells/ml.

<3> Planting of the Cell Suspension and Culture

A cell suspension is planted on a collagen layer and cultured. At this time, the number of cells is adjusted so that a cell layer with a desired cell density can be formed in the end product; the corneal endothelium-like sheet. About 3000 cells/mm² to 7500 cells/mm², (preferably about 5000 cells/mm² to 7500 cells/mm²) are planted so that a cell layer with a cell density of about 2000 cells/mm² to 4000 cells/mm² is formed. The culture is carried out under the same conditions as in the above-mentioned initial culture. The culture time varies depending on the state of cells to be used, but generally it is, for example, 3 to 21 days.

There are no limtitations as to the type of collagen used as a material for the collagen layer; type I, type III, type IV and type VIII collagens can be used. Also, a number of collagens can be used in combination. Such collagens can be extracted and purified, using an acid solubilization, alkali solubilization, and oxygen solubilization method, from the connective tissue of such areas as the skin and cartilage of animals like swine, bovine, sheep, etc. Note that for the purpose of lowering antigenicity, a so-called atherocollagen should be obtained by removing telopeptide via treatment using a catabolic enzyme such as pepsin or trypsin.

It is preferable that, for the collagen layer, a collagen derived from amniotic membrane, from human amniotic membrane in particular, is used. Herein, the collagen layer “derived from amniotic membrane” broadly means that the collagen layer is obtained using amniotic membrane as a starting material. Human amniotic membrane is a membrane covering the outermost layer of the uterus and the placenta, and a basal membrane and an epithelium layer are formed on parenchymal tissue that is rich in collagen. Human amniotic membrane can be collected from human embryonic membrane or placenta at the time of delivery. Specifically, human amniotic membrane can be prepared by treating and purifying the integrated material including human embryonic membrane, placenta, and umbilical cord obtained right after delivery. The method of treating and purifying can be employed using the method described in JP 5 (1993)-56987A. That is to say, amniotic membrane is detached from the embryonic membrane at time of delivery and the remaining tissue is removed by physical treatment such as ultrasonic cleansing and an enzyme treatment. Then, an appropriate cleaning process is carried out and thus the human amniotic membrane can be prepared.

The prepared human amniotic membrane can be cryopreserved before use. The human amniotic membrane can be frozen, for example, in a liquid mixing equal volume ratio of DMEM (Dulbecco's modified Eagle's medium) and glycerol at −80° C. Using cryopreservation, not only an improvement in the operation but also a reduction in antigenicity can be expected.

Intact amniotic membrane may be used as a collagen layer but it is preferable for amniotic membrane, from which the epithelium has been removed by such methods as scraping treatment, to be used. For example, after thawing, cryopreserved human amniotic membrane is subjected to treatment with EDTA or proteolytic enzyme so as to loosen adhesion between the cells and then the epithelium is scraped by using a cell scraper. Thus, the human amniotic membrane from which the epithelium has been removed, can be prepared.

When human amniotic membrane, from which the epithelium has been removed, is used as a collagen layer, it is preferable that corneal endothelial cells are planted at the side of the surface from which the epithelium has been removed and exposed to the surface (i.e., the side of the basement membrane). This is advantageous because it is thought that the side of this surface contains a large amount of type IV collagens and the planted corneal endothelial cells can adhere and proliferate well.

Culture of the corneal endothelial cells on the collagen layer can be carried out, for example, by the following procedures. Firstly, a container with a bottom face made of membrane with a suitable pore size through which culture solution can pass (hereinafter, also referred to as a “culture insert”) is placed with the bottom face faced downward. Then, a collagen layer is formed on the bottom face of the culture insert (inner side of the culture insert). Then, on the collagen layer, the cell suspension is planted and cultured. Note here that the collagen layer may be formed on the bottom face of the culture insert in advance. That is to say, for example, a culture may be carried out by disposing amniotic membrane from which the epithelium has been removed on the bottom face of the culture insert (drying treatment may be carried out once in this state), then setting this culture insert in a culture container, and finally planting and culturing the cell suspension.

An example of a membrane that can be used for the bottom face of the culture insert includes a polycarbonate membrane, having a pore size of about 0.4 μm to 3.0 μm. Besides this, a polyester membrane may be used. Such membranes are widely commercially available.

Centrifugation may be carried out after the cell suspension is planted in the culture insert. Thereby, the cell density on the collagen layer can be increased. Furthermore, adhesion of cells to the surface of the collagen layer becomes excellent. Furthermore, an effect of reducing the deviation of cell density can be obtained. Note here that conditions of centrifugation herein include, for example, 500 rpm (30 g) to 1,000 rpm (70 g) and 1 to 10 minutes.

By culturing, as mentioned above, a corneal endothelium like sheet, in which a cell layer made of cells derived from corneal endothelium is formed on a collagen layer, can be formed. The corneal endothelium-like sheet can be used as a transplantation material (substitute for the corneal endothelium) for treating diseases that require transplantation of the corneal endothelium, such as bullous keratopathy, corneal edema, keratoleukemia and keratoconus. Note here that a sheet obtained by removing part or all of the collagen layer (that is, only the cell layer) may be used as a graft. The collagen layer can be removed by appropriately combining a chemical treatment with EDTA, an enzymatic treatment using proteolytic enzyme, and physical treatment such as scraping using forceps.

Culture (including initial culture, subculture and culture on the collagen layer) of corneal endothelial cells can be carried out in the coexistence of supporting cells. The supporting cell is also referred to as a feeder cell and supplies the culture solution with a growth factor. When the corneal endothelial cells are cultured in the coexistence of supporting cells, improvement in the proliferation efficiency and the promotion corneal endothelial cell differentiation can be expected. With regard to the supporting cells, the likes of 10T1/2 fibroblast (Hyldahl L: Primary cell cultures from human embryonic corneas. J Cell Sci. 66: 343-351, 1984), a 3T3 cell (Swiss mouse 3T3 cell, mouse NIH3T3 cell, 3T3J2 cell or a corneal stromal cell may be used.

When the corneal endothelial cells are cultured in the coexistence of supporting cells, it is preferable for an isolation membrane, with a pore size through which the supporter cells cannot pass, to be provided. This should be between the corneal endothelial cells and the supporting cells. The use of this isolation membrane makes it possible to prevent the supporting cells from entering the side of the corneal endothelial cells at the time of culture. As a result, the supporting cells should not be mixed in the final product; the corneal endothelium-like sheet. This means that a corneal endothelium-like sheet free from immunological rejection problems via the supporting cells, can be constructed. This is extremely clinically significant.

When supporting cells are used, it is preferable for them to be inactivated with mitomycin C. This is advantageous because the inhibition of the proliferation of the corneal endothelial cells, due to the proliferation of the supporting cells themselves, is prevented. Such inactivation may be carried out using radiation treatment.

As for the isolation membrane, a membrane similar to that used in the above-mentioned culture insert can be employed. For example, a membrane with a pore size of about 0.4 μm to 3.0 μm made of polycarbonate or polyester, can be used.

The cell density of the supporting cells may be, for example, about 1×10² cells/cm² or more, preferably in a range from about 1×10² cells/cm² to 1×10⁷ cells/cm², and even more preferably, in a range from about 1×10³ cells/cm² to 1×10⁵ cells/cm². It is not good for the number of supporting cells to be small, because it is thought that the proliferation rate of the corneal endothelial cells is lowered. On the other hand, the number of supporting cells shouldn't be too large, because then the proliferation rate of the corneal endothelial cells is somewhat lowered.

Transplantation methods can include full thickness trepanation and deep keratectomy. In the former method, full thickness cornea is obtained using a trephine and the corneal endothelial cell layer is replaced by the corneal endothelium-like sheet, resulting in the return to a full thickness cornea for the recipient. Specifically, the method can be carried out as follows. Firstly, incision of the full thickness cornea of a recipient (host) is carried out by using a trephine, and a part (or the whole) of the cornea is collected as a button shape. Then, from the piece of cornea collected, the Descemet's membrane and a corneal endothelial cell layer are peeled off. The corneal endothelium-like sheet is then stuck onto the exposed corneal stroma. At this time, the corneal endothelium-like sheet is adhered so that the collagen layer faces the side of the corneal stroma. Thereafter, the corneal graft is returned to the recipient and fixed with a suture.

On the other hand, in the case of deep keratectomy, instead of extracting the full thickness of the cornea, only the deep portion of the cornea is excised. This method is thought to have a reduced burden on behalf of the recipient. Specifically, the method can be carried out as follows. Firstly, a part of the recipient's corneal stroma is delaminated, and the posterior part of corneal stroma and the Descemet's membrane or the endothelial cell layer are excised. Note here that only the endothelial cell layer or only the endothelial cell layer and Descemet's membrane may be peeled off and excised. Next, the corneal endothelium-like sheet is inserted into the excised portion by using a spatula. If necessary, air is pumped into the anterior chamber and the graft is fixed. Furthermore, in the case of a corneal endothelium layer with no collagen layer, only the recipient's affected corneal endothelial cells should be peeled away.

Note that here, it can be confirmed whether or not the transplanted corneal endothelium-like sheet has a barrier function; a pump function can be exhibited, similar to the corneal endothelial cell layer in the living body, by examining the change in corneal thickness after transplantation and the occurrence of edema.

### EXAMPLE 1

### Collection of Amniotic Membrane

Amniotic membrane was obtained during Caesarean section from the placenta of a pregnant woman, who did not have any systemic complications. Sufficient, informed consent was obtained from both the patient and the obstetrician in advance. The operation was carried out cleanly. In accordance with the operation guidelines, the operators washed their hands, and then wore special gowns. Before delivery, a clean vat for obtaining amniotic membrane and physiologic saline for washing were prepared. After delivery, the placenta was transferred to the vat and amniotic membrane tissue was manually removed from the placenta. A portion where amniotic membrane and placenta were strongly adhered to each other, was separated with scissors.

### EXAMPLE 2

### Treatment of Amniotic Membrane

The treatment process of amniotic membrane included: (1) washing, (2) trimming, and (3) storing sequentially, in this order. Throughout these processes, the operation should be carried out in a clean draft. Sterilized containers and instruments were used and in the case of dishes, sterilized disposable ones were used. The amniotic membrane obtained, was washed to remove blood components and further washed in a sufficient amount of physiological saline (0.005% ofloxacin was added). Then, the amniotic membrane was transferred to a phosphate buffer solution (PBS) in a dish and cut and divided into a size of about 4×3 cm with scissors. The divided pieces of amniotic membrane were stored in several dishes filled with a stock solution, and thereafter those amniotic membranes in good condition were selected.

### EXAMPLE 3

### Storage of Amniotic Membrane

1 cc each of stock solution was placed in 2 cc sterilized cryotube and one sheet of amniotic membrane, which had been obtained, washed and selected, was placed and labeled, then stored in a refrigerator at −80° C. For the stock solution, 50% sterilized glycerol in DMEM (Dulbecco's Modified Eagle Medium: GIBCOBRL) was used. The expiration date for use of stored amniotic membrane was determined at 3 months and expired amniotic membrane was disposed of by incineration.

### EXAMPLE 4

### Treatment of Amniotic Epithelium

Amniotic membrane was subjected to treatment to remove the epithelium and was then used for culture. Firstly, amniotic membrane stored at −80° C. was thawed at room temperature, and then washed thoroughly in a dish of sterilized phosphate buffer solution (PBS). After washing, the amniotic membrane was stored in a 0.02% EDTA solution (Nacalai tesque) at 37° C. for 2 hours, and then the epithelium was mechanically scraped out by using a cell scraper (Nunc, USA) and used as a substrate for culture. Note that it was confirmed that one layer of the amniotic epithelium was completely scraped using this procedure by optical microscope and electron microscope (scanning electron microscope) operations.

### EXAMPLE 5

### Collection and Initial Culture of Human Corneal Endothelial Cells

The Descemet's membrane and an endothelial cell layer were peeled off from the stroma of human (age: 47, male) corneal tissue by using a forceps, then disposed on a 35 mm culture dish, and treated with 1 ml of dispase (final concentration: 1.2 U/ml) that had been double diluted with PBS containing no calcium under conditions of 37° C. and in 5% CO₂ for 60 minutes. With this treatment, many of the corneal endothelial cells dropped off from the Descemet's membrane. In order to aid the dropping off of the corneal endothelial cells residing in the Descemet's membrane, pipetting treatment was carried out. Next, the Descemet's membrane was removed and the corneal endothelial cells were transferred to a 15 cc centrifugation tube. A culture solution was added to the centrifugation tube to make a total amount of 5 ml and then centrifugation was carried out under conditions of 1000 rpm, 70 g for 3 minutes. For the culture solution, DMEM (GIBCOBRL) was used, to which 10% FBS (Dainippon Pharmaceutical Co., Ltd), 2 ng/ml b-FGF (Invitrogen), penicillin (50 U/ml)-streptomycin (50 μg/ml) (Nacalai tesque) were added. Supernatant was removed and then culture solution was added to form about 1 ml of cell suspension. This cell suspension was planted on a 24-hole culture dish coated with type IV collagen and cultured under conditions of 37° C. and in 5% CO₂. Thereafter, the culture solutions were exchanged for new solution every 48 hours.

### EXAMPLE 6

### Subculture of Endothelial Cells

When the cultured cells became confluent, the culture solution was removed and the cells were washed three times with 1 ml of PBS containing no calcium. After washing, 200 μl of 0.05% trypsin-EDTA was added and placed under conditions of 37° C. and 5% CO₂ for 3 minutes. With this treatment, endothelial cells, adhered to the bottom surface of the culture dish, were allowed to float. 5 ml of cultured solutions were added and the cell suspension was transferred to a 15 cc centrifugation tube and centrifugation was carried out under conditions of 1000 rpm, 70 g for 3 minutes. Supernatant was removed and then culture solution was added to form about 2 ml of cell suspension. This cell suspension was planted on a 35 mm culture dish coated with type IV collagen and cultured under conditions of 37° C. and 5% CO₂. Thereafter, the culture solutions were exchanged for new solutions every 48 hours. When the cells became confluent, the same treatment as mentioned above was carried out, and the subculture was repeated until the necessary number of cells were obtained. When the subculture was repeated 5 times, the cell density was measured. The cell density was 500 to 1000 cells/mm². Note that by carrying out a subculture using the above-mentioned treatments, at least 10 generations of subculture were achievable.

### EXAMPLE 7

### Production of High Density Cultured Corneal Endothelial Cells

The subcultured cells were treated with 0.05% trypsin-EDTA and then the number of cells was counted. Subsequently, centrifugation was carried out and supernatant was removed. Then, a culture solution was added so that the cell density became 2.0×10⁶ cells/ml. By using the obtained cell suspension, a corneal endothelial cell layer was produced via the following procedures. As a culture instrument, a 6-hole culture dish (Corning, N.Y.) and a culture insert (culture insert: culture inserting container; made of polycarbonate; average pore size: 3.0 μm; produced by Corning, N.Y.) were used. For a culture solution, the same culture solution mentioned above was used.

Firstly, a culture insert was disposed in a culture dish. Then, cell suspension was planted in the culture insert so that the cell density reached about 5000 cells/mm². Thereafter, the culture dish was centrifuged under conditions of 1000 rpm, 70 g for 3 minutes. After centrifugation, culture was carried out under conditions of 37° C. and 5% CO₂. As a result, after culture for 14 days, a cell layer with a cell density of about 3000 cells/mm² was obtained. Furthermore, in a 2-week culture, no morphological changes in cells were observed.

Note here that FIG. 5 is a cross-sectional view schematically showing the state during culturing. It shows a state in which the culture insert (2) is disposed in a culture dish (1), and the corneal endothelial cells (3) are cultured in the culture insert 2. Reference numeral 5 denotes a culture medium.

### EXAMPLE 8

### Production of a Corneal Endothelium-Like Sheet Using Amniotic Membrane as a Substrate

By using a cell suspension (cell density: about 2.0×10⁶ cells/ml), which is also used in Example 7, a corneal endothelium-like sheet was produced via the following procedures. For culture instruments, the same instruments as in Example 7 were used.

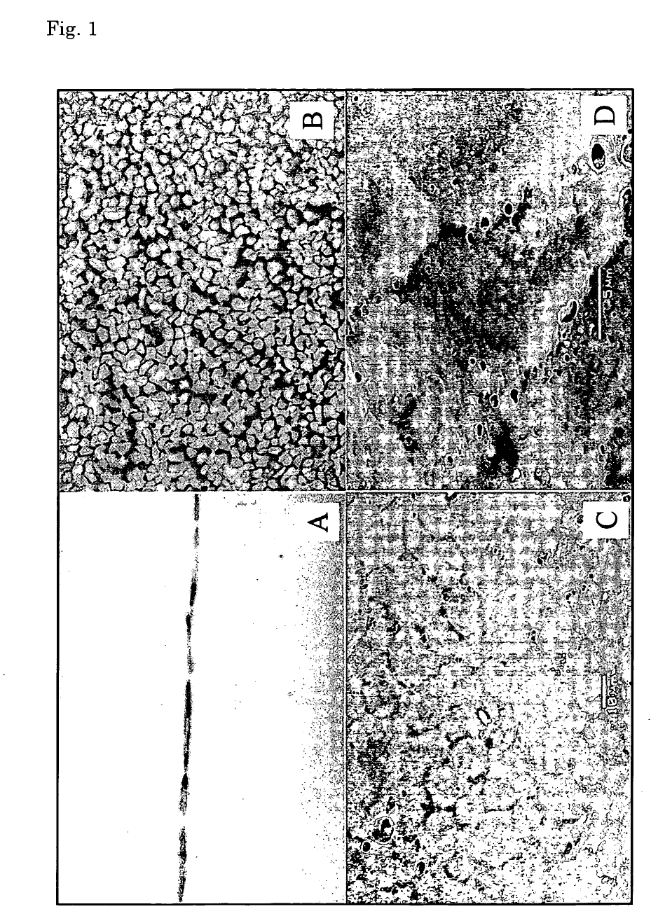

Firstly, a culture insert was disposed in a culture dish. Then, amniotic membrane from which the epithelium had been scraped out, obtained in Example 4, was extended and laid with the side from which the epithelium had been scraped out facing upward. Subsequently, cell suspension was planted on the amniotic membrane so that the cell density reached about 5000 cells/mm². Thereafter, the culture dish was centrifuged under conditions of 1000 rpm, 70 g for 3 minutes. After centrifugation, culture was carried out under conditions of 37° C. and 5% CO₂ for about 14 days. As a result, as in the living body, a continuous single-structured cell layer was constructed (FIG. 1A). When the cell density of the cell layer was measured, it was 3340 cells/mm², which showed a similar cell density to that of the cell layer obtained in Example 7 (FIG. 1B). In addition, when the morphology was observed by using a scanning electron microscope, cells constituting the cell layer had a hexagonal, flat shape, similar to the endothelial cells of the living body (FIGS. 1C and 1D). Furthermore, it was observed that a cell layer was configured in which these hexagonal-shaped cells were arranged more or less uniformly.

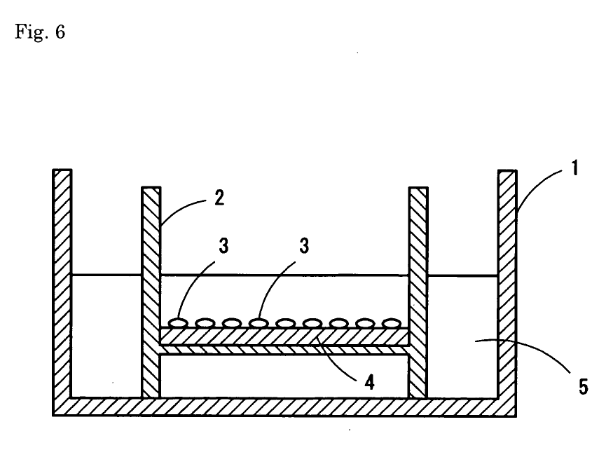

Note that FIG. 6 is a cross-sectional view schematically showing the conditions during culturing. In a culture dish (1), a culture insert (2) is disposed. Furthermore, it shows that on the bottom face of the culture insert (2), amniotic membrane (4) is extended and the corneal endothelial cells (3) are cultured thereon. Reference numeral 5 denotes a culture medium.

### EXAMPLE 9

### Transplantation of the Corneal Endothelium-Like Sheet

a. Transplantation by Full Thickness Trepanation.

Full thickness of the central portion of the cornea (host cornea) in a rabbit (aged 6-weeks, Japanese white color species) was incised with a trephine of 7 to 8 mm in diameter. The Descemet's membrane of the host corneal graft obtained in a button shape was peeled off, including the endothelial cell layer. The corneal endothelium-like sheet obtained in Example 8 was taken out from the culture dish with a forceps, followed by incision with a 6 to 8 mm trephine and placed on the stroma of the host cornea from which the Descemet's membrane and endothelial cell had been removed. At this time, a suture was not used and the corneal endothelium-like sheet was adhered to the corneal stroma by absorbing water with a sponge and drying slightly. The host corneal graft in which the corneal endothelium was disposed on amniotic membrane was sutured to the host cornea with 10-0 nylon thread. Thereafter, every day until day 7 after transplantation, the thickness of the transplanted portion of the cornea and the state of the edema were observed. Note that as a control for comparison, a group (AM group) using amniotic membrane instead of the corneal endothelium-like sheet and a group (control group), to which a host corneal graft, from which the Descemet's membrane with endothelial cells had been removed, were used.

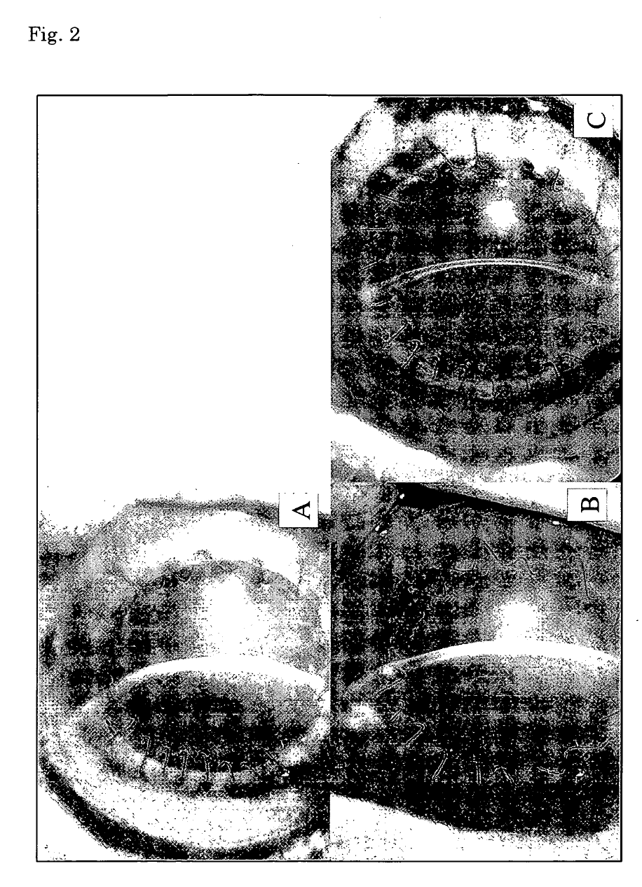

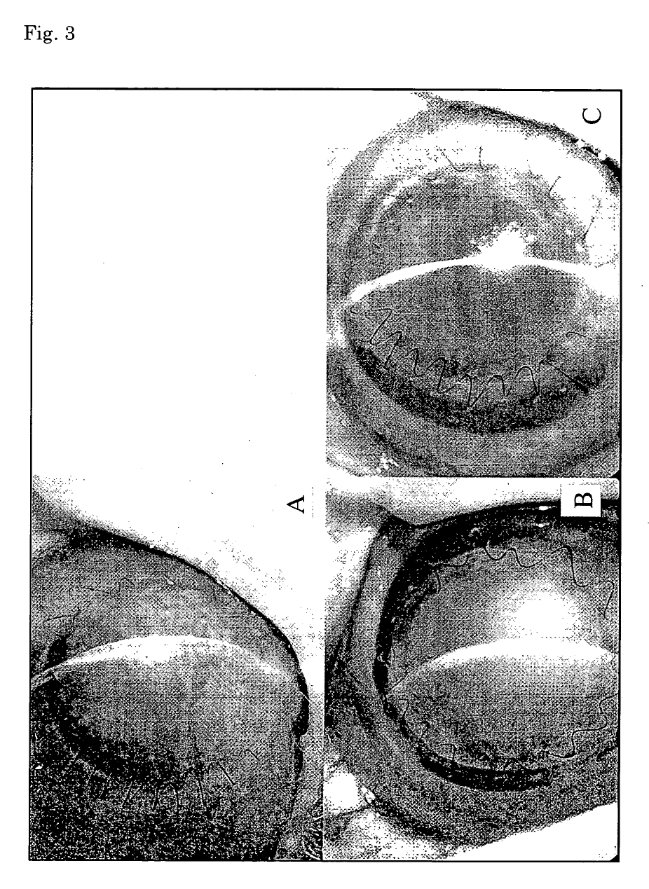

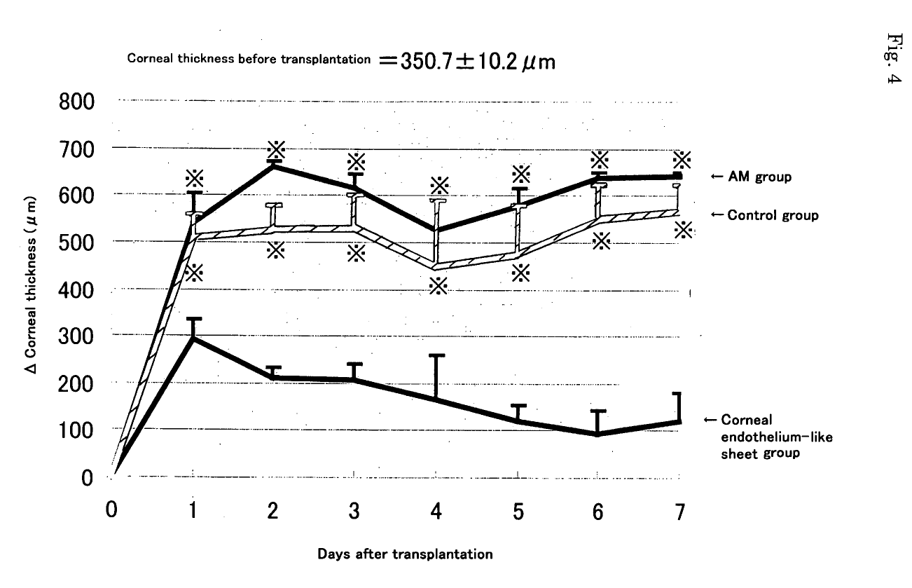

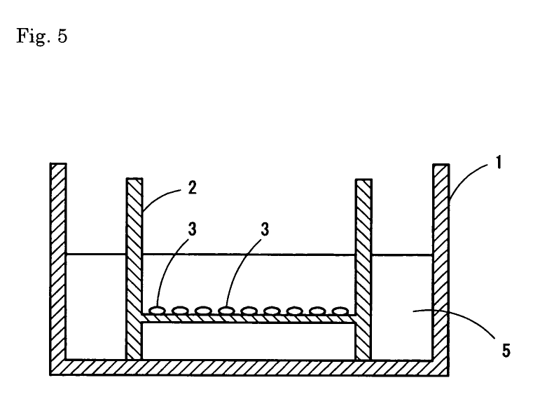

FIG. 2 shows the state of the transplanted site at day 4 after transplantation. In the group in which the Descemet's membrane was removed, and transplantation was carried out (control group), significant edema was observed (FIG. 2A). Furthermore, also in the AM group, as in the control group, significant edema was observed (FIG. 2B). On the other hand, in the cornea to which the corneal endothelium-like sheet was transplanted, compared with the control and AM groups, edema was significantly reduced and transparency remained. At the time of observation, day 7 after transplantation, in the cornea to which the corneal endothelium-like sheet was transplanted, as compared with the control group (FIG. 3A) and the AM group (FIG. 3B), edema was reduced and transparency was maintained (FIG. 3C). In addition, in the cornea to which the corneal endothelium-like sheet was transplanted, the corneal thickness was significantly reduced as compared with the control and AM groups (FIG. 4).

The above mentioned results suggest that, on amniotic membrane, cells derived from the human corneal endothelial cells had a morphology that was extremely similar to that of the cells in a living body. Furthermore, by using centrifugation, it was possible to obtain a cell density of about 3000 cells/mm². It was also shown that the corneal endothelium-like sheet produced by this technique could perform its function in vivo.

b. Transplantation by Deep Keratectomy

The corneal (host cornea) stroma of a rabbit (aged 6-weeks, Japanese white color species) was peeled away and a deep layer of the parenchyma stroma, with a diameter of 7 to 8 mm in the central portion, was incised, including the endothelial cell layer. Alternately, only the endothelial cells and the Descemet's membrane, with a 7 to 8 mm diameter in the central part, were peeled off and removed from the anterior chamber. The corneal endothelium-like sheet obtained in Example 8 was inserted into the excised portion using a spatula, from between the stromal layers or into the anterior chamber. Then, by pumping air into the anterior chamber, the graft was fixed. Thereafter, every day until day 7 after transplantation, the thickness of the transplanted portion of the cornea and the state of edema were observed. As a result, in the cornea to which the corneal endothelium-like sheet was transplanted, as in full thickness trepanation, the corneal thickness and corneal edema were significantly reduced compared to the AM group to which only amniotic membrane was transplanted or the control group in which only removal of the corneal endothelium was carried out.

From the examples mentioned above, it was confirmed that the corneal endothelium-like sheet performed its function in vivo. That is to say, it has been confirmed that the corneal endothelium-like sheet produced, functions excellently as a substitute for corneal endothelium and can be used as a transplantation material for reconstructing the corneal endothelium when the corneal endothelium is damaged or affected. Furthermore, it has been proven that by using this sheet, an ideal operating technique capable of minimizing the failure of the immunosuppressive environment in the anterior ocular segment and replacing only the damaged corneal endothelial cells with healthy endothelial cells, can be achieved.

The present invention is not limited to the description of the above embodiments. A variety of modifications, which are within the scopes of the following claims and which are easily achieved by a person skilled in the art, are included in the present invention.

### INDUSTRIAL APPLICABILITY

This present invention; the corneal endothelium-like sheet, has a structure similar to the corneal endothelial cell layer of the living body and can be used as a transplantation material for treating various diseases that require corneal endothelial cell transplantation. The corneal endothelium-like sheet has excellent survival properties following transplantation, performs the functions of the corneal endothelial cell layer, i.e., a barrier function and a pump function, extremely useful in the reconstruction of the damaged corneal endothelial cell layer. Meanwhile, this corneal endothelium-like sheet is formed by culturing and proliferating the collected cornea endothelial cells in vitro. Therefore, it is possible to produce a transplantation material based on a small amount of corneal endothelial cells. Also in a patient with a reduced number of corneal endothelial cells, transplantation materials can be produced from auto-corneal endothelial cells. This means that corneal transplantation that does not cause rejection can be realized. Furthermore, a corneal endothelium transplantation technique in which only the damaged site is replaced, can be realized. An ideal operating technique that does not cause complications during and after transplantation, which was a problem in conventional penetrating keratoplasty, can now be made available.

1. A corneal endothelium-like sheet, comprising:
a collagen layer; and
a cell layer formed on the collagen layer, the cell layer consisting of cells derived from corneal endothelium.

2. A corneal endothelium-like sheet according to claim 1, wherein the collagen layer is derived from amniotic membrane.

3. A corneal endothelium-like sheet according to claim 1, wherein the collagen layer consists of amniotic membrane from which the epithelium has been removed.

4. A corneal endothelium-like sheet comprising a cell layer consisting of cells derived from corneal endothelium.

5. A corneal endothelium-like sheet according to claim 1, wherein the cell layer has a monolayer structure.

6. A corneal endothelium-like sheet according to claim 1, wherein the cell density of the cell layer is about 2000 cells/mm² to about 4000 cells/mm².

7. A corneal endothelium-like sheet according to claim 1, wherein the plane view shape of the cells derived from corneal endothelium is hexagonal.

8. A corneal endothelium-like sheet according to claim 1, wherein the cells derived from corneal endothelium are arranged regularly in the cell layer.

9. A method for constructing a corneal endothelium-like sheet, the method comprising the following steps:
a) culturing and proliferating collected corneal endothelial cells;
b) collecting the proliferated corneal endothelial cells and producing a cell suspension; and
c) planting the cell suspension on a collagen layer and culturing thereof.

10. A method for producing a corneal endothelium-like sheet according to claim 9, wherein the following step is carried out after step b):
b-1) increasing the cell density in the cell suspension using centrifugation.

11. A method for producing a corneal endothelium-like sheet according to claim 9, wherein centrifugation is carried out after planting the cell suspension in step c).

12. A method for producing a corneal endothelium-like sheet according to claim 9, wherein step c) comprises the following steps:
c-1) placing a container in a culture container, the container having a bottom surface consisting of a membrane with a pore size capable of allowing a culture solution to pass through;
c-2) forming a collagen layer on the bottom face of the container;
c-3) planting the cell suspension on the collagen layer;
c-4) carrying out centrifugation;
c-5) culturing.

13. A method for producing a corneal endothelium-like sheet according to claim 9, wherein the collagen layer is derived from amniotic membrane.

14. A method for producing a corneal endothelium-like sheet according to claim 9, wherein the collagen layer consists of amniotic membrane from which the epithelium has been removed.
